SEQUENCE LISTING (30 sequences)

SEQ ID NO: 1 - DNA, 20 bp, Artificial Sequence
  Catalase_Forward
gccacaggaaagtacccctc

SEQ ID NO: 2 - DNA, 20 bp, Artificial Sequence
  Catalase_Reverse
cggtgagtgtcagcataggc

SEQ ID NO: 3 - DNA, 20 bp, Artificial Sequence
  SOD_Forward
atggcgacgaaggccgtgtg

SEQ ID NO: 4 - DNA, 20 bp, Artificial Sequence
  SOD_Reverse
gaccaccagtgtgcggccaa

SEQ ID NO: 5 - DNA, 20 bp, Artificial Sequence
  GPx_Forward
tgggcatcaggagaacgcca

SEQ ID NO: 6 - DNA, 20 bp, Artificial Sequence
  GPx_Reverse
tgcgtaggggcacaccgtca

SEQ ID NO: 7 - DNA, 20 bp, Artificial Sequence
  COL1A1_Forward
cacgacaaagcagaaacatc

SEQ ID NO: 8 - DNA, 21 bp, Artificial Sequence
  COL1A1_Reverse
acacatcaagacaagaacgag

SEQ ID NO: 9 - DNA, 20 bp, Artificial Sequence
  COL3A1_Forward
tggtgcccctggtccttgct

SEQ ID NO: 10 - DNA, 20 bp, Artificial Sequence
  COL3A1_Reverse
tacggggcaaaaccgccagc

SEQ ID NO: 11 - DNA, 20 bp, Artificial Sequence
  COL4A1_Forward
tcctggcctccagggaatta

SEQ ID NO: 12 - DNA, 20 bp, Artificial Sequence
  COL4A1_Reverse
atcaacagatggggtgcctg

SEQ ID NO: 13 - DNA, 22 bp, Artificial Sequence
  COL7A1_Forward
ctgggagagaaggtcgtgatgg

SEQ ID NO: 14 - DNA, 20 bp, Artificial Sequence
  COL7A1_Reverse
tccacattcggcacacttcc

SEQ ID NO: 15 - DNA, 22 bp, Artificial Sequence
  MMP-1_Forward
aagtcaagtttgtggcttatgg

SEQ ID NO: 16 - DNA, 24 bp, Artificial Sequence
  MMP-1_Reverse
gactcatgtctcctgtctctttct

SEQ ID NO: 17 - DNA, 22 bp, Artificial Sequence
  MMP-2_Forward
catacaaagggattgccaggac

SEQ ID NO: 18 - DNA, 20 bp, Artificial Sequence
  MMP-2_Reverse
atcgctccagacttggaagg

SEQ ID NO: 19 - DNA, 20 bp, Artificial Sequence
  MMP-9_Forward
tctatggtcctcgccctgaa

SEQ ID NO: 20 - DNA, 20 bp, Artificial Sequence
  MMP-9_Reverse
catcgtccaccggactcaaa

SEQ ID NO: 21 - DNA, 20 bp, Artificial Sequence
  MMP-13_Forward
ctatggtccaggagatgaag

SEQ ID NO: 22 - DNA, 20 bp, Artificial Sequence
  MMP-13_Reverse
agagtcttgcctgtatcctc

SEQ ID NO: 23 - DNA, 20 bp, Artificial Sequence
  IL-1b_Forward
agccatggcagaagtacctg

SEQ ID NO: 24 - DNA, 20 bp, Artificial Sequence
  IL-1b_Reverse
tccatggccacaacaactga

SEQ ID NO: 25 - DNA, 20 bp, Artificial Sequence
  IL-6_Forward
atgaggagacttgcctggtg

SEQ ID NO: 26 - DNA, 20 bp, Artificial Sequence
  IL-6_Reverse
acaacaatctgaggtgccca

SEQ ID NO: 27 - DNA, 20 bp, Artificial Sequence
  IL-8_Forward
ccaggaagaaaccaccggaa

SEQ ID NO: 28 - DNA, 20 bp, Artificial Sequence
  IL-8_Reverse
cctctgcacccagttttcct

SEQ ID NO: 29 - DNA, 20 bp, Artificial Sequence
  GAPDH_Forward
ctcctgttcgacagtcagcc

SEQ ID NO: 30 - DNA, 20 bp, Artificial Sequence
  GAPDH_Reverse
tcgccccacttgattttgga

(19) United States
(12) Patent Application Publication
(10) Pub. No.: US 2023/0000758 A1
(43) Pub. Date: Jan. 5, 2023

(54) METHOD FOR PREVENTING SKIN AGING COMPRISING KOREAN MINT EXTRACT

(71) Applicants: COSMAX NBT, INC., Seongnam-si (KR); COSMAX NS, INC., Seongnam-si (KR)

(72) Inventors: Jin Hee YOO, Seoul (KR); Su Young CHOI, Yongin-si (KR); Won Il YUN, Seoul (KR)

(73) Assignees: COSMAX NBT, INC., Seongnam-si (KR); COSMAX NS, INC., Seongnam-si (KR)

(21) Appl. No.: 17/358,113

(22) Filed: Jun. 25, 2021

(51) Int. Cl. A61K 8/9789; A61Q 19/08; A61Q 19/00; A23L 33/105

(52) U.S. Cl. CPC A61K 8/9789; A61Q 19/08; A61Q 19/004; A23L 33/105; A61K 2236/331; A61K 2236/35; A61K 2800/92; A23V 2002/00

(57) ABSTRACT

Provided are a method for preventing or treating photoaging of skin, including: administering a composition comprising an effective amount of an extract of Agastache rugosa O Kuntze to a subject in need thereof. The composition decreases expression of matrix metalloproteinases (MMPs) induced by ultraviolet B (UVB) irradiation; decreases expression of mitogen-activated protein kinase (MAPK)/activator protein-1 (AP-1) signaling; improves collagen synthesis; decreases skin inflammation induced by NF-κB; and decreases oxidative stress induced by UVB irradiation.

### SEQUENCE LISTING

The instant application contains a Sequence Listing which has been submitted in ASCII format via EFS-Web and is hereby incorporated by reference in its entirety. Said ASCII copy is named GBLO035_Sequence Listing.txt, created on 06/29/2021 and is 6,221 bytes in size.

### TECHNICAL FIELD

The disclosure relates to a method for preventing or treating photoaging of skin comprising administering a composition comprising an effective amount of an extract of Agastache rugosa O Kuntze.

### BACKGROUND

Skin aging involves intrinsic and extrinsic aging (Clin Dermatol. 2013. 31:750-758). Intrinsic aging occurs with the passage of time, whereas extrinsic aging occurs owing to exposure to external factors, such as harmful chemicals, air pollutants, and ultraviolet B (UVB) irradiation (J Eur Acad Dermatol Venereol. 2011. 25:873-884). Extrinsic aging resulting from long-term exposure to UVB irradiation is called photoaging. During this process, UVB irradiation penetrates the dermis and induces breakdown of skin components; this is associated with skin inflammation, DNA damage, and oxidative stress (Toxicol Appl Pharmacol. 2004. 195:298-308). In particular, excessive generation of reactive oxygen species (ROS), a major element involved in stimulating oxidative stress, damages skin cells, which degrades and disorganizes extracellular matrix (ECM) components (J Agric Food Chem. 2015. 63:5428-5438).

UVB irradiation-induced ROS production activates mitogen-activated protein kinases (MAPKs), subsequently stimulating to formation of the activator protein-1 (AP-1) (J Am Acad Dermatol. 2006. 55:1-19; Int J Dermatol. 2013. 52:531-543; and PLoS One. 2016. 11: e0159998). AP-1 increases the expression of matrix metalloproteinases (MMPs), which stimulate breakdown of ECM components especially collagens (Antioxid Redox Signal. 2014. 21:1063-1077). Degrading collagens, main components involved in supporting dermal layers, lead to the destruction and collapse of skin structure (Am J Pathol. 2004. 165:741-751; and Mol Med Rep. 2015. 11:3344-3348). Therefore, downregulating MMP expression is an effective strategy to prevent and delay the development of photoaging-related symptoms, such as wrinkle formation and thickening in UVB-exposed skin (Exp Dermatol. 2010. 19:e182-e190). In terms of new collagen formation in the ECM, AP-1 complex blocks type-I procollagen production and downregulates collagen gene expression in UVB-exposed skin cells (Ageing Res Rev. 2015. 21:16-29). Thus, photoaging is closely associated with both stimulating collagen breakdown and blocking collagen formation by suppressing type-I collagen synthesis and collagen gene expression (Phytomedicine. 2016. 23:1273-1284). Additionally, exposure to UVB irradiation enhances translocation of nuclear factor kappa-B (NF-κB) to the nucleus where it is mainly involved transcription of inflammatory cytokines (Int J Cosmet Sci. 2005. 27:17-34). The inflammatory responses induced by activation of NF-κB in UVB-exposed skin cells lead to MMP overexpression and collagen degradation (J Photochem Photobiol B. 2015. 144:35-41).

Agastache rugosa O Kuntze, also known as Korean mint, is mainly found in Korea, Japan, and China. Agastache rugosa O Kuntze has been used as a food source and in traditional medicines to cure disease because it has varied bioactivities, which encompass anti-oxidant, anti-melanogenic, anti-atherogenic, anti-inflammatory, and anti-fungal properties (FEBS Lett. 2001. 495:142-147; and J Funct Foods. 2017. 30:30-38). Previously, A. rugosa leaf extract was shown to reduce photoaging by activating glutathione and superoxide dismutase (SOD) in human keratinocytes (HaCaT) (J Photochem Photobiol B. 2016. 163:170-176). However, the antiphotoaging effect of Agastache rugosa O Kuntze extract (ARE) and the underlying mechanisms in human dermal fibroblasts (HS68) have not yet been proven. Here, we determined the attenuating effect of ARE on photoaging in UVB-treated human dermal fibroblasts by examining the underlying molecular mechanisms.

### SUMMARY

An aspect of the present invention provides a method for preventing or treating photoaging of skin, the method comprising: administering a composition comprising an effective amount of an extract of Agastache rugosa O Kuntze to a subject in need thereof.

In an aspect, the extract of Agastache rugosa O Kuntze is obtained by using water or an organic solvent.

In an aspect, the organic solvent is selected from the group consisting of methanol, ethanol, propanol, isopropanol, butanol, acetone, ether, benzene, chloroform, ethyl acetate, methylene chloride, hexane, and cyclohexane.

In an aspect, the photoaging is due to ultraviolet B (UVB) irradiation.

In an aspect, the composition decreases expression of matrix metalloproteinases (MMPs) such as MMP-1, MMP-2, MMP-9, and MMP-13, which is induced by UVB irradiation.

In an aspect, the composition decreases expression of mitogen-activated protein kinase (MAPK)/activator protein-1 (AP-1) signaling such as p-extracellular signal-regulated kinase (ERK), ERK, p-c-Jun N-terminal kinase (JNK), JNK, p-p38, and p38.

In an aspect, the composition improves collagen synthesis and increase expression of collagen type I alpha 1 (COL1A1), collagen type II alpha 1 (COL3A1), collagen type IV alpha 1 (COL4A1), and collagen type VII alpha 1 (COL7A1).

In an aspect, the composition decreases skin inflammation induced by NF-κB.

In an aspect, the composition decreases oxidative stress induced by UVB irradiation.

In an aspect, the composition is functional food composition.

In an aspect, the composition ranges 0.1 mg/kg to 100 mg/kg.

### BRIEF DESCRIPTION OF THE DRAWINGS

FIG. 1A shows effect of Agastache rugosa O Kuntze extract (ARE) on reactive oxygen species (ROS) production in Ultraviolet B (UVB)-treated HS68 cells (##P<0.01 compared with the control group; and **P<0.01 compared with the UVB-treated cells).

FIG. 1B shows reverse transcription-polymerase chain reaction analysis of the expression of catalase, superoxide dismutase (SOD), and glutathione peroxidase (GPx) after ARE (10 and 20 μg/mL) treatment. Glyceraldehyde-3-phosphate dehydrogenase (GAPDH) was used as a housekeeping gene. Data are expressed as mean±SD of three independent experiments (##P<0.01 compared with the control group; and *P<0.05 and **P<0.01 compared with the UVB-treated cells).

FIG. 2A shows effect of Agastache rugosa O Kuntze extract (ARE) on ultraviolet B (UVB)-induced matrix metalloproteinase (MMP) expression by reverse transcription-polymerase chain reaction analysis of the expression of matrix metalloproteinase (MMP)-1, MMP-2, MMP-9, and MMP-13 after ARE (10 and 20 μg/mL) treatment. Glyceraldehyde-3-phosphate dehydrogenase (GAPDH) was used as housekeeping genes. Data are expressed as mean±SD of three independent experiments (##P<0.01 compared with the control group; and *P<0.05 and **P<0.01 compared with the UVB-treated cells).

FIG. 2B shows effect of Agastache rugosa O Kuntze extract (ARE) on ultraviolet B (UVB)-induced mitogen-activated protein kinase (MAPK)/activator protein-1 (AP-1) signaling by western blot analysis of the expression of p-extracellular signal-regulated kinase (ERK), ERK, p-c-Jun N-terminal kinase (JNK), JNK, p-p38, and p38 after ARE (10 and 20 μg/mL) treatment. α-Tubulin was used as housekeeping genes. Data are expressed as mean±SD of three independent experiments (##P<0.01 compared with the control group; and *P<0.05 and **P<0.01 compared with the UVB-treated cells).

FIG. 2C shows effect of Agastache rugosa O Kuntze extract (ARE) on ultraviolet B (UVB)-induced mitogen-activated protein kinase (MAPK)/activator protein-1 (AP-1) signaling by western blot analysis of the expression of p-c-Jun, c-Jun, and c-Fos after ARE (10 and 20 μg/mL) treatment. α-Tubulin was used as housekeeping genes. Data are expressed as mean±SD of three independent experiments (##P<0.01 compared with the control group; and *P<0.05 and **P<0.01 compared with the UVB-treated cells).

FIG. 3A shows effect of Agastache rugosa O Kuntze extract (ARE) on collagen synthesis by reverse transcription-polymerase chain reaction analysis of the expression of collagen type I alpha 1 (COL1A1), collagen type III alpha 1 (COL3A1), collagen type IV alpha 1 (COL4A1), and collagen type VII alpha 1 (COL7A1) after ARE (10 and 20 μg/mL) treatment. Glyceraldehyde-3-phosphate dehydrogenase (GAPDH) was used as a housekeeping gene. Data are expressed as mean±SD of three independent experiments (##P<0.01 compared with the control group; and **P<0.01 compared with the UVB-treated cells).

FIG. 3B shows effect of Agastache rugosa O Kuntze extract (ARE) on collagen synthesis by type-I procollagen production by analyzing type-I procollagen content after ARE (10 and 20 μg/mL) treatment (##P<0.01 compared with the control group; and **P<0.01 compared with the UVB-treated cells).

FIG. 4A shows effect of Agastache rugosa O Kuntze (ARE) on inflammatory cytokine levels by western blot analysis of the expression of nuclear factor kappa B (NF-κB) after ARE (10 and 20 μg/mL) treatment. α-Tubulin was used as housekeeping genes. Data are expressed as mean±SD of three independent experiments. (##P<0.01 compared with the control group; and **P<0.01 compared with the UVB-treated cells).

FIG. 4B shows effect of Agastache rugosa O Kuntze extract (ARE) on inflammatory cytokine levels by reverse transcription-polymerase chain reaction analysis of the expression of IL-1p, IL-6, and IL-8 after ARE (10 and 20 μg/mL) treatment. GAPDH was used as housekeeping genes. Data are expressed as mean±SD of three independent experiments. (##P<0.01 compared with the control group; and **P<0.01 compared with the UVB-treated cells).

### DETAILED DESCRIPTION OF THE EMBODIMENTS

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the aspects provided herein, because the scope of the aspects provided herein is limited only by the claims. Unless otherwise defined, all technical and scientific terms used herein have the same meaning as commonly understood by one of ordinary skill in the art to which aspects provided herein belong.

The term “treating” or “treatment” as used herein comprises a treatment relieving, decrease, reducing or alleviating at least one symptom in a human patient or effecting a delay of progression of a disease. For example, treatment can be the diminishment of one or several symptoms of a disorder or complete eradication of a disorder. Within the meaning of the present disclosure, the term “treat” also denotes to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject.

The term “prevent”, “preventing” or “prevention” as used herein comprises the prevention of at least one symptom associated with or caused by the state, disease or disorder being prevented.

The term “pharmaceutically effective amount” or “clinically effective amount” of a composition is an amount sufficient to provide an observable improvement over the baseline clinically observable signs and symptoms of the disorder treated with the combination.

The term “photoaging” is the characteristic changes to skin induced by chronic UVA and UVB exposure.

An aspect of the present invention provides a method for preventing or treating photoaging of skin, the method comprising: administering a composition comprising an effective amount of an extract of Agastache rugosa O Kuntze to a subject in need thereof.

Agastache rugosa O Kuntze may be used as those obtained by extracting and isolating from nature using an extraction and separation method known in the art, and the “extract” defined in the present invention may be obtained from Agastache rugosa O Kuntze using a suitable solvent and include, for example, a hot-water extract, a polar solvent-soluble extract, or a non-polar solvent-soluble extract of Agastache rugosa O Kuntze.

As a suitable solvent for extracting an extract from Agastache rugosa O Kuntze, any solvent acceptable in the art may be used, and water or an organic solvent may be used. For example, various solvents such as purified water, alcohols having 1 to 4 carbon atoms, such as methanol, ethanol, propanol, isopropanol, and butanol, acetone, ether, benzene, chloroform, ethyl acetate, methylene chloride, hexane, cyclohexane, etc. maybe, but not limited to, used alone or in combination.

As the extraction method, any of methods such as hot water extraction method, cold-immersion extraction method, reflux cooling extraction method, solvent extraction method, steam distillation method, ultrasonic extraction method, dissolution method, and compression method can be selected and used. Further, the desired extract may be further subjected to a conventional fractionation process, and may be purified using a conventional purification method. There is no limitation on the production method of the extract of Agastache rugosa O Kuntze of the present invention, and any known method can be used.

For example, the extract of Agastache rugosa O Kuntze in the composition of the present invention is such that the first extract extracted with the hot water extraction method or the solvent extraction method can be prepared in a powder state by an additional process such as vacuum distillation, freeze drying, or spray drying is. Further, the additional purified fraction can be obtained, in which the first extract is treated using various chromatographies such as silica gel column chromatography, thin layer chromatography, and high-performance liquid chromatography.

Therefore, in the present invention, the extract of Agastache rugosa O Kuntze is a concept including all the extracts, fractions and purifications obtained in each step of extraction, fractionation, or purification, their diluted solutions, concentrates, or dried products.

In an embodiment, the extract of Agastache rugosa O Kuntze may be obtained by using water or an organic solvent. The organic solvent may be selected from the group consisting of methanol, ethanol, propanol, isopropanol, butanol, acetone, ether, benzene, chloroform, ethyl acetate, methylene chloride, hexane, and cyclohexane.

In an embodiment, the photoaging may be due to ultraviolet B (UVB) irradiation.

In an embodiment, the composition may decrease expression of matrix metalloproteinases (MMPs) induced by UVB irradiation. In an exemplary embodiment, the composition decreases MMP-1, MMP-2, MMP-9, and MMP-13 induced by UVB irradiation.

The term “gene expression” or “expression” is understood to refer to transcription of a DNA sequence, translation of an mRNA transcript, and secretion of a protein product or a fragment thereof.

In an embodiment, the composition may decrease expression of mitogen-activated protein kinase (MAPK)/activator protein-1 (AP-1) signaling. In an exemplary embodiment, the composition decreases p-extracellular signal-regulated kinase (ERK), ERK, p-c-Jun N-terminal kinase (JNK), JNK, p-p38, and p38.

In an embodiment, the composition may improve collagen synthesis. In an exemplary embodiment, the composition increases expression of collagen type I alpha 1 (COL1A1), collagen type III alpha 1 (COL3A1), collagen type TV alpha 1 (COL4A1), and collagen type VII alpha 1 (COL7A1).

In an embodiment, the composition decreases skin inflammation induced by NF-κB and oxidative stress induced by UVB irradiation.

In an embodiment, the composition is functional food composition.

The functional food of the present invention may be manufactured and processed in the form of tablets, capsules, powders, granules, liquids, rings, etc. for the purpose of prevention and treatment of photoaging.

The term “functional food” refers to foods prepared and processed using raw materials or ingredients having useful functions, which means that it is ingested for the purpose of obtaining a beneficial effect for health use such as control of nutrients or physiological action for the structure and function of the human body.

The functional food of the present invention may comprise conventional food additives. Examples of the items listed in the above-mentioned “Food Additives Codex” include chemical compounds such as ketone, glycine, calcium citrate, nicotinic acid, and cinnamic acid; natural additives such as persimmon color, licorice extract, crystalline cellulose, kaoliang color, and guar gum; mixture preparations such as L-glutamic acid sodium preparations, alkali agents for noodles, preservative preparations, and tar coloring preparations.

For example, the extract of Agastache rugosa O Kuntze, an active ingredient of the present invention, is mixed with the excipient, binder, disintegrant, and other additives, then the mixture is granulated in a usual manner, and then a lubricant and the like is added and the granulate is compression-molded, or then the mixture can be directly compression-molded, thereby obtaining the health functional food in the form of tablet. In addition, the health functional food in the tablet form may contain a flavoring agent or the like as needed.

The hard capsule of the capsule-type functional food can be prepared by filling a conventional hard capsule with a mixture of the extract of Agastache rugosa O Kuntze, an active ingredient of the present invention, with an additive such as an excipient. The soft capsule may be prepared by filling a capsule base such as gelatin with a mixture of the extract of Agastache rugosa O Kuntze, an active ingredient of the present invention, with an additive such as an excipient. The soft capsule may contain plasticizers such as glycerin and sorbitol, coloring agents, preservatives and the like, if necessary.

The ring-type functional food can be prepared by molding a mixture of the extract of Agastache rugosa O Kuntze, an active ingredient of the present invention with excipients, binders, disintegrants, and the like, according to a known method. If necessary, it may be coated with white sugars or other coating aids, or the surface thereof may be coated with a material such as starch and talc.

The granule-type functional food can be prepared by a conventionally known method in which a mixture of the extract of Agastache rugosa O Kuntze, an active ingredient of the present invention with excipients, binders, and disintegrants is formed into granules. If necessary, fragrance agents, flavoring agents, etc. can be added.

The functional food may be a beverage, a meat, a chocolate, a foodstuff, a confectionery, a pizza, a ramen, a noodle, a gum, a candy, an ice cream, an alcoholic beverage, a vitamin complex, a health supplement food, etc.

In an embodiment, the extract of Agastache rugosa O Kuntze is included in an amount of 0.001% to 20% by weight based on the total weight of the composition. In an embodiment, the extract of Agastache rugosa O Kuntze is included in an amount of 0.01% to 10% by weight based on the total weight of the composition.

In an embodiment, the composition may be administered in a range from about 0.001 mg/kg to about 100 mg/kg. In an embodiment, the composition may be administered in a range from about 0.01 mg/kg to about 10 mg/kg. In another embodiment, the composition may be administered in a range from about 0.1 mg/kg to about 10 mg/kg.

### EXAMPLES

Hereinafter, the present disclosure is explained in detail by Examples. The following Examples are intended to further illustrate the present invention without limiting its scope.

### Example 1. Materials and Methods

### 1.1. Preparation of Agastache rugosa Extract (ARE)

The dried aerial parts of Agastache rugosa O Kuntze were extracted with water. ARE filtrates were evaporated. Cell culture and UVB irradiation HS68 cells were purchased from the American Type Culture Collection (Manassas, Va., USA). The cells were maintained in Dulbecco's modified Eagle's medium (HyClone Laboratories, Inc., Logan, Utah, USA) containing 120 U/mL penicillin and 75 μg/mL streptomycin (Invitrogen, Grand Island, N.Y., USA), and 10% fetal bovine serum (HyClone Laboratories, Inc.) in a humidified atmosphere of 5% CO2 at 37° C. To induce photoaging, the cells were exposed to UVB irradiation (15 mJ/cm2) by using a UV crosslinker CL-1000M (Ultra-Violet Products Ltd., Cambridge, UK).

### 1.2. Measurement of ROS Production

After treatment with ARE and UVB exposure, cells were incubated with 2,7-dichlorofluorescein diacetate (Sigma-Aldrich Co.) in a CO2 incubator at 37° C. for 30 min. Subsequently, the cellular fluorescence was recorded using a SpectraMax Gemini EM microplate spectrofluorometer (Molecular Devices Inc.).

### 1.3. Western Blot Analysis

Cells were lysed with NP40 lysis buffer (Elpis-Biotech, Daejeon, Korea) supplemented with 0.2% protease inhibitor cocktail (Sigma-Aldrich Co.). After centrifugation at 16,000 g at 4° C. for 15 min, the concentration of protein in the supernatant was evaluated by Bradford solution (Bio-Rad Laboratories, Inc., Hercules, Calif., USA) with bovine serum albumin (bioWORLD, Dublin, Ohio, USA) used as a standard. Equal amounts of proteins in each group were subjected to 8˜-10% sodium dodecyl sulfate polyacrylamide gel electrophoresis for 90 min at 110 V. The separated proteins were transferred onto nitrocellulose blotting membranes (GE Healthcare, Piscataway, N.J., USA) for 1 h at 110 V. After nonspecific sites were blocked with 5% skimmed milk (Difco Laboratories Inc., Detroit, Mich., USA), membranes were immunoblotted with primary antibodies against NF-κB, phospho (p)-p38, extracellular signal-regulated kinase (p-ERK), p-c-Jun N terminal kinases (p-JNK), and c-Fos (Santa Cruz Biotechnology Inc., Santa Cruz, Calif., USA); and p-c-Jun, c-Jun, ERK, JNK, p38, and α-tubulin (Cell Signaling Technology, Danvers, Mass., USA) at room temperature for 4 h. After washing three times in Tris-buffered saline (Dynebio, Gyeonggi, Korea) containing Tween 20, membranes were incubated with horseradish peroxidase-conjugated goat anti-rabbit or anti-mouse secondary antibodies (Bethyl Laboratories, Montgomery, Tex., USA) at room temperature for 1 h. Enhanced chemiluminescence solution (Amersham BioSciences UK Ltd., Little Chalfont, UK) was used to develop the membranes. The band of protein was visualized using a G:BOX EF imaging system (Syngene, Cambridge, UK) and the GeneSys program.

### 1.4. Measurement of Type-I Procollagen Contents

After treatment with ARE and UVB exposure, the type-I procollagen content of the medium was evaluated using a human procollagen I alpha 1 enzyme-linked immunosorbent assay kit (Abcam, Cambridge, Mass., USA), according to the company's protocol. Absorbance was recorded using a VERSAmax tunable microplate reader (Molecular Devices Inc.) at 450 nm. Reverse transcription-polymerase chain reaction (RT-PCR) RNAiso Plus (Takara Bio Inc., Kusatsu, Japan) was used for isolation of total RNA from cells. The quantity and quality of the isolated mRNA were spectrophotometrically determined using a NanoDrop Lite spectrophotometer (Thermo Fisher Scientific Inc., Waltham, Mass., USA). The mRNA was reverse-transcribed to cDNA with RT PreMix (Elpis Biotech, Daejeon, Korea). Next, the cDNA of target genes was amplified using specific primers and HiPi PCR PreMix (Elpis Biotech). The list of primer sequences is shown in Table 1. Amplification was carried out over 25-30 cycles; denaturation for 30 s at 94° C., annealing for 1 min at 56-60° C., and extension for 1 min at 72° C. PCR experiments were carried out using a SimpliAmp Thermal Cycler (Applied Biosystems, Hercules, Calif., USA). The amplified cDNA was stained with Loading star (Dynebio) and separated for 30 min in a Mupid electrophoresis chamber (Advance, Tokyo, Japan) on 1.5% agarose gel. The bands of target genes were visualized with G:BOX EF imaging system (Syngene) and GeneSys program.

TABLE 1

| | | | SEQ |
|---|---|---|---|
| | | | ID |
| Gene | Primer | Sequence (5’→3’) | NO: |
| Catalase | Forward | GCCACAGGAAAGTACCCCTC | 1 |
| | Reverse | CGGTGAGTGTCAGCATAGGC | 2 |
| SOD | Forward | ATGGCGACGAAGGCCGTGTG | 3 |
| | Reverse | GACCACCAGTGTGCGGCCAA | 4 |
| GPx | Forward | TGGGCATCAGGAGAACGCCA | 5 |
| | Reverse | TGCGTAGGGGCACACCGTCA | 6 |
| COL1A1 | Forward | CACGACAAAGCAGAAACATC | 7 |
| | Reverse | ACACATCAAGACAAGAACGAG | 8 |
| COL3A1 | Forward | TGGTGCCCCTGGTCCTTGCT | 9 |
| | Reverse | TACGGGGCAAAACCGCCAGC | 10 |
| COL4A1 | Forward | TCCTGGCCTCCAGGGAATTA | 11 |
| | Reverse | ATCAACAGATGGGGTGCCTG | 12 |
| COL7A1 | Forward | CTGGGAGAGAAGGTCGTGATGG | 13 |
| | Reverse | TCCACATTCGGCACACTTCC | 14 |
| MMP-1 | Forward | AAGTCAAGTTTGTGGCTTATGG | 15 |
| | Reverse | GACTCATGTCTCCTGTCTCTTTCT | 16 |
| MMP-2 | Forward | CATACAAAGGGATTGCCAGGAC | 17 |
| | Reverse | ATCGCTCCAGACTTGGAAGG | 18 |
| MMP-9 | Forward | TCTATGGTCCTCGCCCTGAA | 19 |
| | Reverse | CATCGTCCACCGGACTCAAA | 20 |
| MMP-13 | Forward | CTATGGTCCAGGAGATGAAG | 21 |
| | Reverse | AGAGTCTTGCCTGTATCCTC | 22 |
| IL-1β | Forward | AGCCATGGCAGAAGTACCTG | 23 |
| | Reverse | TCCATGGCCACAACAACTGA | 24 |
| IL-6 | Forward | ATGAGGAGACTTGCCTGGTG | 25 |
| | Reverse | ACAACAATCTGAGGTGCCCA | 26 |
| IL-8 | Forward | CCAGGAAGAAACCACCGGAA | 27 |
| | Reverse | CCTCTGCACCCAGTTTTCCT | 28 |
| GAPDH | Forward | CTCCTGTTCGACAGTCAGCC | 29 |
| | Reverse | TCGCCCCACTTGATTTTGGA | 30 |

SOD, superoxide dismutase; GPx, glutathione peroxidase; COL1A1, collagen type I alpha 1; COL3A1, collagen type III alpha 1; COL4A1, collagen type IV alpha 1; COL7A1, collagen type VII alpha 1; MMP, matrix metalloproteinase; IL, interleukin; GAPDH, glyceraldehyde-3-phosphate dehydrogenase.

### 1.5. Statistical Analysis

All experiments were carried out three or more times. Data are shown as the mean±standard deviation (SD) and were analyzed using one-way analysis of variance (ANOVA). Intergroup differences were evaluated by Scheffe's test using SPSS 24.0 (SPSS Inc., Chicago, Ill., USA). Statistical significance is indicated by P<0.05 or P<0.01. Hash tags and asterisks denote significant differences compared to control cells and UVB-treated cells, respectively.

### Example 2. ARE Suppresses ROS Production and Increases Anti-Oxidant Enzyme Expression

Excessive production of ROS is a major cause of photoaging in UVB-irradiated skin, and can further induce oxidative stress and MMP expression (Photodermatol Photoimmunol Photomed. 2014. 30:237-245). Thus, inhibition of UVB-induced ROS production could be an effective strategy to prevent photoaging. The ability of ARE to scavenge free radicals, and thereby exert anti-oxidant effects, was previously reported (Biomed Chromatogr. 2016. 30:225-231). Moreover, the phytochemicals, acacetin, and rosmarinic acid, which are present in ARE, have been reported to possess anti-oxidant activities (Free Radic Biol Med. 1990. 9:19-21; and J Korean Soc Agric Chem Biotechnol. 1999. 42:262-266).

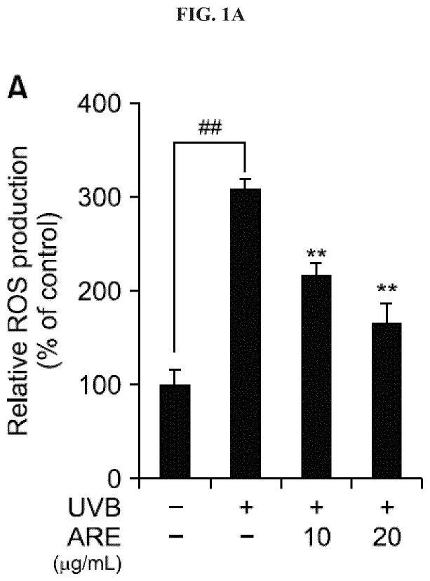

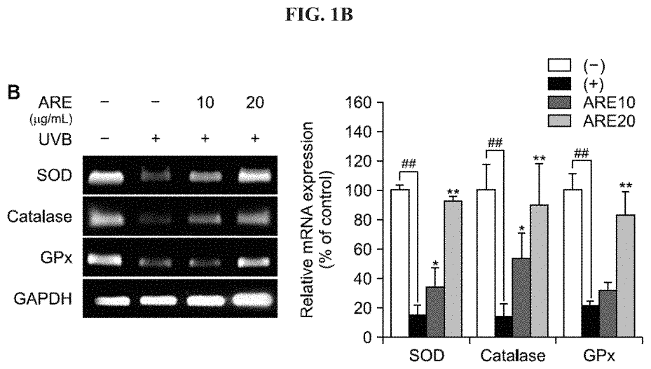

Based on these studies, we evaluated the anti-oxidant properties of ARE in UVB-exposed human dermal fibroblasts by examining total levels of intracellular ROS. UVB irradiation markedly increased ROS generation; however, ARE treatment dose-dependently alleviated ROS production by 92% (10 μg/mL) and 142% (20 μg/mL), compared with UVB-treated HS68 cells (FIG. 1A). These results indicate that ARE exhibits anti-oxidant activity by suppressing UVB-induced ROS production.

We also examined mRNA expression of anti-oxidant enzymes in UVB-treated HS68 cells. It was reported that UVB-induced ROS led to cellular senescence in dermal fibroblasts (Yang and Li, 2015). As anti-oxidant enzymes protect skin from UVB-induced thickening and wrinkle formation, it may be necessary to enhance the activities of these enzymes to prevent photoaging (J Cosmet Dermatol. 2007. 6:2-6). According to a previous study, linarin, an active compound in ARE, markedly increased expression of the major anti-oxidant enzyme catalase in lipopolysaccharide-stimulated mouse lung tissues (Int J Mol Med. 2018. 42:1460-1472). We evaluated mRNA expression of the anti-oxidant enzymes catalase, SOD, and glutathione peroxidase (GPx) in cells treated with ARE. UVB irradiation reduced expression of all three transcripts; however, mRNA expression levels of these enzymes were markedly elevated after ARE treatment (FIG. 1B).

Taken together, the reduction of UVB-stimulated ROS production and elevated expression of anti-oxidant enzymes suggest that ARE has potent anti-oxidant properties.

### Example 3. ARE Suppresses UVB-Induced MMP Expression Through the Downregulation of the MAPK/AP-1 Pathway

UVB irradiation stimulates MAPKs, which promotes heterodimerization of proteins in the AP-1 complex. Enhancement of the MAPK/AP-1 signaling pathway promotes upregulation of MMP expression, which subsequently results in breakdown of ECM components, such as elastic fibers, glycosaminoglycans, and collagens. Consequently, this signaling cascade results in photoaging related phenotypes, including wrinkle formation, epidermal thickening, and skin dryness (Food Chem Toxicol. 2012. 50:4260-4269). It was reported that UVB exposure upregulated the pro-MMP-2 and -9 expression; however, probiotic-fermented ARE decreased the MMP expression in UVB-induced HaCaT cells (BMC Complement Altern Med. 2018. 18:196).

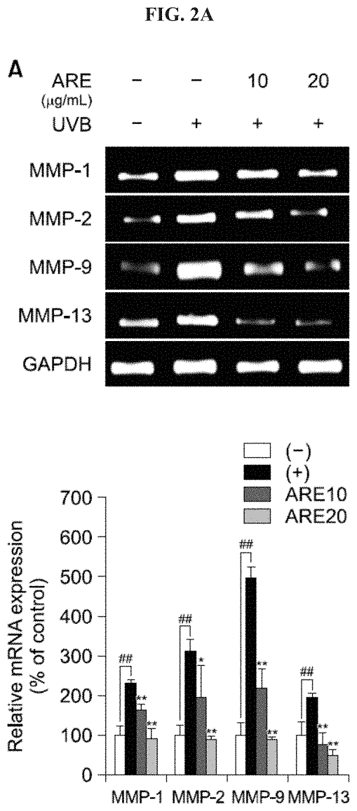

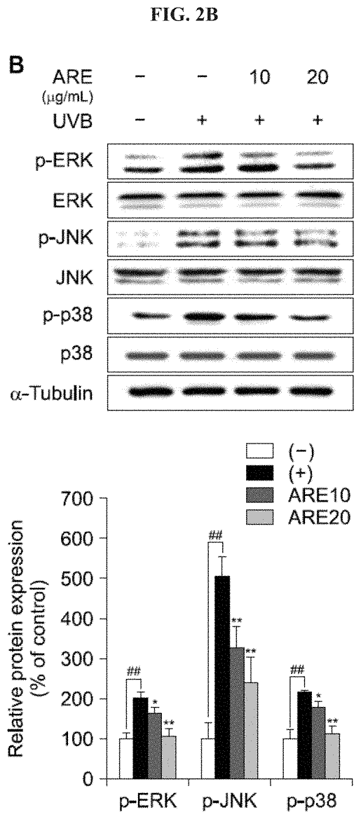

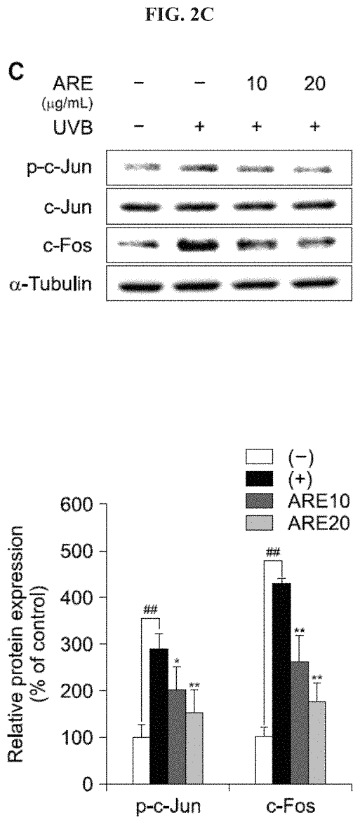

Based on this effect of ARE, we evaluated the expression of MMPs, MAPK, and AP-1 in UVB-irradiated HS68 cells. UVB treatment markedly increased MMP mRNA expression; however, treatment with ARE significantly decreased their expression (FIG. 2A). In this study, the MAPK signaling pathway was activated in the UVB-exposed cells. However, ARE significantly decreased MAPK expression, compared with UVB-irradiated HS68 cells (FIG. 2B). In comparison with untreated cells, UVB irradiation significantly upregulated protein expression of the AP-1 components p-c-Jun and c-Fos, whereas ARE downregulated p-c-Jun and c-Fos (FIG. 2C).

Collectively, these data indicate that ARE decreases UVB-induced MMPs expression by inactivating the MAPK/AP-1 signaling pathway.

### Example 4. ARE Increases Type-I Procollagen Production and Collagen Expression

UVB irradiation stimulates collagen breakdown by increasing the expression of MMPs and downregulates expression of collagen genes by activating the AP-1 complex (Mol Med Rep. 2015. 11:3344-3348). These responses alter the ECM architecture, resulting in wrinkle formation, elastosis, and skin dryness (Ageing Res Rev. 2015. 21:16-29).

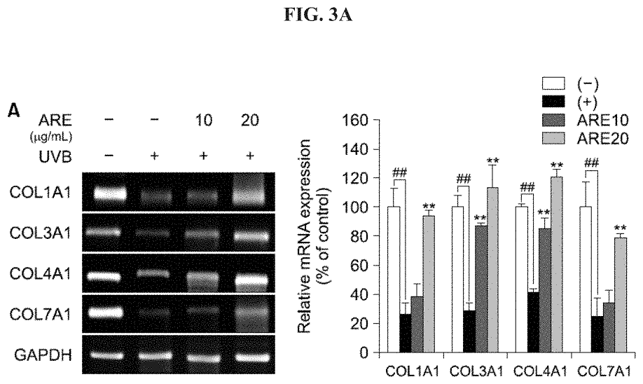

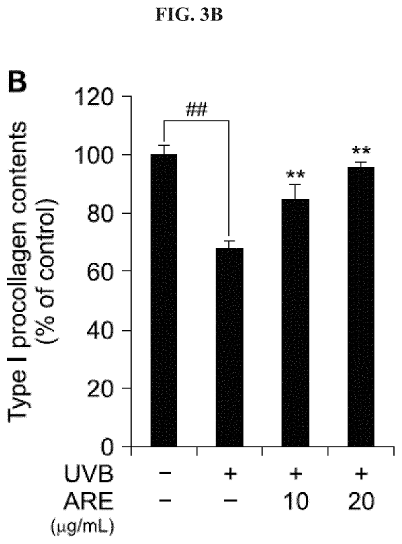

Since ARE was revealed to reduce MMP expression by inhibiting MAPK/AP-1 cell signaling in UVB-treated HS68 cells, we evaluated the effect of ARE on regulation of genes related to collagen synthesis and production of type-I procollagen. The expression of the collagen genes [collagen type I al (COL1A1), collagen type III al (COL3A1), collagen type IV al (COL4A1), and collagen type VII al (COL7A1)] was significantly reduced in UVB-treated HS68 cells. However, ARE markedly upregulated mRNA expression of collagen genes (FIG. 3A). ARE treatment prevented UVB irradiation from reducing type-1 procollagen production in a dose-dependent manner. Specifically, at a dose of 20 μg/mL, ARE increased type-I procollagen content by 27% (FIG. 3B). Consistent with these current results, A. rugosa recovered skin barrier function by increasing expression of collagens in atopic dermatitis induced mice (J East-West Nurs Res. 20:57-62).

This study demonstrates that ARE attenuates skin photoaging by activating type-I procollagen production and improving collagen synthesis in UVB-irradiated HS68 cells.

### Example 5. ARE Inhibits UVB-Induced Inflammation

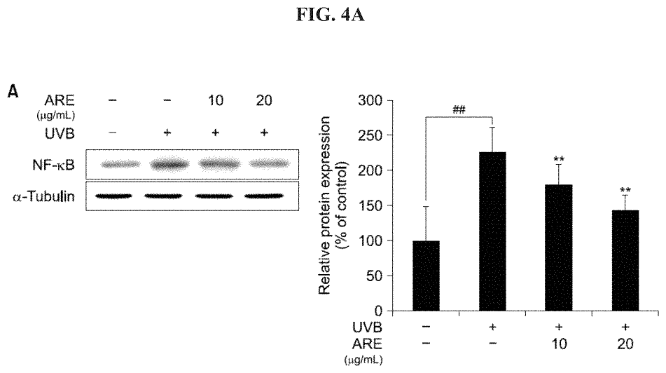

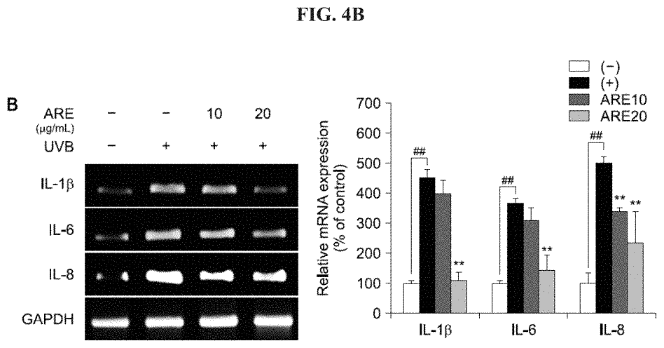

Previous study showed that UVB irradiation elevated inflammatory responses by activating NF-κB and stimulating expression of NF-κB-mediated inflammatory cytokines (J Pathol. 2008. 214:149-160). UVB-induced inflammatory responses upregulate MMP expression, resulting in skin dehydration, psoriasis, and erythema (Free Radic Biol Med. 2012. 52:1734-1743). Here, mRNA expression of inflammatory mediators was examined in UVB-exposed HS68 cells. UVB irradiation elevated protein expression of NF-κB, which was inhibited by ARE (FIG. 4A). In addition, mRNA expression of NF-κB-stimulated inflammatory cytokines was decreased in cells treated with ARE, compared with UVB-irradiated HS68 cells (FIG. 4B).

The anti-inflammatory effect of ARE was reported in an osteosarcoma cell line, accompanied by downregulation of interleukin-1β and tumor necrosis factor-α (Arch Pharm Res. 2005. 28:305-310). Consistent with this report, our data showed that ARE exhibited strong anti-inflammatory effects, thereby playing a critical role in anti-photoaging property by suppressing expression of NF-κB and pro-inflammatory cytokines. Taken together, these results suggest that ARE could be used as an anti-photoaging agent owing to its alleviation of UVB-induced skin inflammatory responses.

In this study, the anti-photoaging effect of ARE was investigated in UVB-irradiated human dermal fibroblasts. ARE suppressed UVB-induced oxidative stress by reducing production of ROS and by increasing expression of anti-oxidant enzymes. In addition, ARE attenuated UVB-activated MAPK/AP-1 signaling, resulting in downregulation of MMP expression. Moreover, ARE both upregulated expression of collagen genes and increased production of procollagen. Further, ARE alleviated UVB-stimulated inflammatory responses by suppressing expression of inflammatory cytokines. Collectively, our findings suggest that ARE could be a potential candidate for skin anti-photoaging treatment. To develop ARE as a novel anti-photoaging agent, potentially as an ingredient in functional foods or nutraceuticals, the anti-photoaging effect of ARE should be verified in animal studies and clinical trials.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

What is claimed is:

1. A method for preventing or treating photoaging of skin, comprising:
administering a composition comprising an effective amount of an extract of Agastache rugosa O Kuntze to a subject in need preventing or treating the photoaging of skin.

2. The method of claim 1, wherein the extract of Agastache rugosa O Kuntze is obtained by using water or an organic solvent.

3. The method of claim 2, the organic solvent is selected from the group consisting of methanol, ethanol, propanol, isopropanol, butanol, acetone, ether, benzene, chloroform, ethyl acetate, methylene chloride, hexane, and cyclohexane.

4. The method of claim 1, the photoaging is due to ultraviolet B (UVB) irradiation.

5. The method of claim 1, wherein the composition decreases an expression of matrix metalloproteinases (MMPs) induced by ultraviolet B (UVB) irradiation.

6. The method of claim 1, wherein the composition decreases an expression of mitogen-activated protein kinase (MAPK)/activator protein-1 (AP-1) signaling.

7. The method of claim 1, wherein the composition improves collagen synthesis.

8. The method of claim 1, wherein the composition decreases skin inflammation induced by NF-κB.

9. The method of claim 1, wherein the composition decreases oxidative stress induced by ultraviolet B (UVB) irradiation.

10. The method of claim 1, wherein the composition is functional food composition.

11. The method of claim 1, wherein the extract of Agastache rugosa O Kuntze is included in an amount of 0.001% to 20% by weight based on total weight of the composition.
